SEQUENCE LISTING (12 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 4 aa, artificial sequence
  Furin cleavage site substituting motif
GSAS

SEQ ID NO: 3 - protein, 1179 aa, Severe acute respiratory syndrome coronavirus 2
SSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVA

SEQ ID NO: 4 - protein, 27 aa, artificial sequence
  T4 fibritin trimerization motif
GYIPEAPRDGQAYVRKDGEWVLLSTFL

SEQ ID NO: 5 - protein, 8 aa, artificial sequence
  Twin-Strep-Tag. repeat
WSHPQFEK

SEQ ID NO: 6 - protein, 8 aa, artificial sequence
  HRV3C protease cleavage site
LEVLFQGP

SEQ ID NO: 7 - protein, 1208 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 8 - protein, 1288 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 9 - DNA, 3870 bp, Severe acute respiratory syndrome coronavirus 2
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctgggtctgcttcctctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggaccctccagaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagggatccggatacatccccgaggcccccagagatggccaggcctacgtgcggaaggacggcgagtgggtactgctgagcacattcctgggcagatccctggaggtgctgttccagggcccaggccatcaccaccatcaccaccatcatagcgcctggtcccacccccagttcgagaagggcggcggtagtggagggggcggatctggcggctcagcttggagccacccccagttcgaaaagtgataa

SEQ ID NO: 10 - DNA, 23 bp, artificial sequence
  Leader-specific primer sgLeadSARSCoV2-F
cgatctcttgtagatctgttctc

SEQ ID NO: 11 - DNA, 22 bp, artificial sequence
  E-Sarbeco-R primer
atattgcagcagtacgcacaca

SEQ ID NO: 12 - DNA, 26 bp, artificial sequence
  E-Sarbeco probe
  misc_feature (1)..(1): Covalent link to hexachlorofluorescein
  misc_feature (26)..(26): Covalent link to BHQ1 quencher
acactagccatccttactgcgcttcg

(19) United States
(12) Patent Application Publication
(10) Pub. No.: US 2024/0002446 A1
(43) Pub. Date: Jan. 4, 2024

(54) PROTEOLIPOSOMES COMPRISING A SARS-COV-2 S GLYCOPROTEIN ECTODOMAIN AND THEIR USE AS A VACCINE

(71) Applicants: UNIVERSITÉ GRENOBLE ALPES, SAINT MARTIN D'HÈRES (FR); CENTRE NATIONAL DE LA RECHERCHE SCIENTIFIQUE, PARIS (FR); COMMISSARIAT À L'ENERGIE ATOMIQUE ET AUX ÉNERGIES ALTERNATIVES, PARIS (FR); CENTRE HOSPITALIER UNIVERSITAIRE GRENOBLE ALPES, LA TRONCHE (FR)

(72) Inventors: Winfried WEISSENHORN, SEYSSINS (FR); Guidenn SULBARAN MACHADO, GRENOBLE (FR); Delphine GUILLIGAY, VOREPPE (FR); Gregory EFFANTIN, SEYSSINS (FR); Axelle AMEN, GRENOBLE (FR); Pascal POIGNARD, GRENOBLE (FR); Roger LE GRAND, CHATENAY-MALABRY (FR); Pauline MAISONNASSE, GENTILLY (FR)

(21) Appl. No.: 17/853,541

(22) Filed: Jun. 29, 2022

(51) Int. Cl. C07K 14/005; A61K 9/127; A61P 31/14

(52) U.S. Cl. CPC C07K 14/005; A61K 39/00; A61P 31/14; A61K 9/1275

(57) ABSTRACT

A recombinant SARS-CoV-2 S glycoprotein ectodomain trimer is disclosed, including three recombinant protomers each containing at least the SARS-CoV-2 S glycoprotein ectodomain, and wherein: in each protomer, the furin cleavage site is inactivated/disrupted; Arg408 of one of the protomers is covalently linked to Lys378 of another one of the protomers; and Lys947 of one of the protomers is covalently linked to Arg1019 and/or to Lys776 of another one of the protomers.

### BACKGROUND OF THE INVENTION

### Field of the Invention

The invention relates to the field of preventing and/or treating a coronavirus infection, in particular a SARS-CoV-2 infection.

More particularly, the invention relates to a recombinant SARS-CoV-2 S glycoprotein ectodomain trimer stabilized in the native conformation, as well as a proteoliposome comprising such a recombinant trimer and a vaccine based on such a proteoliposome. The invention also relates to a method of treating or preventing a SARS-CoV-2 infection in a subject using such a vaccine.

### Description of the Related Art

The severe acute respiratory syndrome coronavirus 2, SARS-CoV-2, a beta-coronavirus, is the etiological agent of coronavirus disease 2019 (COVID-19), which quickly developed into a worldwide pandemic causing more than 5 million deaths as of November 2021 and highlighting the urgent need for effective infection control and prevention.

An important correlate of protection of antiviral vaccines is the generation of neutralizing antibodies. The main SARS-CoV-2 target for inducing neutralizing antibodies is the spike (S) glycoprotein, which plays an essential role in virus attachment, fusion and entry into host cells, in particular due to its surface location. The S glycoprotein is composed of the S1 subunit that harbors the receptor-binding domain (RBD) and the S2 membrane fusion subunit that anchors the S trimer in the virus membrane.

RBD binding to the cellular receptor Angiotensin-converting enzyme 2 (ACE2) leads to virus attachment and subsequent S2-mediated fusion with endosomal membranes establishes infection. The S glycoprotein is synthesized as a trimeric precursor polyprotein that is proteolytically cleaved by furin and furin-like proteases in the Golgi generating the non-covalently linked S1-S2 heterotrimer. The structure of the S glycoprotein reveals a compact heterotrimer composed of S1 (NTD, RBD, RBM and two subdomains), S2 (the transmembrane region) and a cytoplasmic domain. The conformation of RBD is in a dynamic equilibrium between either all RBDs in a closed, receptor-inaccessible conformation or one or two RBDs in the “up”, receptor-accessible, conformation. Only S RBD in the ‘up’ position allows receptor binding, which triggers the S2 post fusion conformation in proteolytically cleaved S glycoprotein (Yan et al. (2020). Science 367, 1444-1448; Lan et al. (2020). Nature 581, 215-220).

Antibodies targeting the S glycoprotein were identified upon SARS-CoV-2 seroconversion, which mostly target RBD that is immunodominant (Piccoli et al. (2020). Cell 183, 1024-1042). This led to the isolation of many neutralizing antibodies, which confirmed antibody-based vaccination strategies. Many of these antibodies have been shown to provide in vivo protection against SARS-Cov-2 challenge in small animals and nonhuman primates or are in clinical development and use (Weinreich et al. (2021). N Engl J Med 384, 238-251).

The magnitude of antibody responses to S glycoprotein during natural infection varies greatly and correlates with disease severity and duration. Basal responses are generally maintained for months or decline within weeks after infection, notably in asymptomatic individuals. Thus, any vaccine-based approach aims to induce long-lasting immunity.

A number of animal models have been developed to study SARS CoV-2 infection including the macaque model, which demonstrated induction of innate, cellular and humoral responses upon infection conferring partial protection against reinfection (Deng et al. (2020). Science 369, 818-823). Consequently, many early vaccine candidates provided protection in the macaque model including the currently licensed vaccines based on S-specific mRNA delivery (BNT162b2, Pfizer/BioNTech; mRNA-1273, Moderna), adenovirus vectors (ChAdOx1 nCoV-19, Oxford/AstraZeneca; Ad26.COV2.S, Johnson & Johnson) and inactivated SARS-CoV-2 (PiCoVacc/CoronaVac, Sinovac). Employing the classical subunit approach, S glycoprotein subunit vaccine candidates have generated different levels of neutralizing antibody responses in preclinical testing (Liang et al. (2021). Nat Commun 12, 1346).

### SUMMARY OF THE INVENTION

The inventors have now discovered that a recombinant SARS-CoV-2 S glycoprotein ectodomain trimer comprising specific modifications, when integrated in synthetic virus-like particles employing liposomes, efficiently protects animals to which it is administered from infections by at least wild-type SARS-CoV-2 and Alpha pseudovirus variants, and neutralizes Beta and Gamma pseudovirus variants at reduced potency, by providing sterilizing immunity, more particularly by eliciting mucosal immune responses. Moreover, RBD-specific antibodies are predominant after a first and second immunization, but, after a third immunization median S-specific ED50s are 3 times higher than RBD-specific ED50s, suggesting that more than two immunizations allow to expand the reactive B cell repertoire that target non-RBD S epitopes, which proves particularly advantageous in the field of development of vaccines against native SARS-CoV-2 and variants thereof.

More particularly, an object of the invention is a recombinant SARS-CoV-2 S glycoprotein ectodomain trimer comprising three recombinant protomers each containing at least the SARS-CoV-2 S glycoprotein ectodomain, wherein, at least:
in each protomer, the furin cleavage site, situated at positions 682 to 685 in the amino acid sequence of the native SARS-CoV-2 S glycoprotein (SEQ ID No: 1), is inactivated/disrupted;
the amino acid residue located at position 408 in the amino acid sequence of the native SARS-CoV-2 S glycoprotein (SEQ ID No: 1) of one of said protomers is covalently linked to the amino acid residue located at position 378 in the amino acid sequence of the native SARS-CoV-2 S glycoprotein (SEQ ID No: 1) of another one of said protomers; and
the amino acid residue located at position 947 in the amino acid sequence of the native SARS-CoV-2 S glycoprotein (SEQ ID No: 1) of one of said protomers is covalently linked to the amino acid residue located at position 1019 in the amino acid sequence of the native SARS-CoV-2 S glycoprotein (SEQ ID No: 1) of another one of said protomers and/or the amino acid residue located at position 947 in the amino acid sequence of the native SARS-CoV-2 S glycoprotein (SEQ ID No: 1) of one of said protomers is covalently linked to the amino acid residue located at position 776 in the amino acid sequence of the native SARS-CoV-2 S glycoprotein (SEQ ID No: 1) of another one of said protomers.

The trimer having these characteristics is advantageously stabilized in the native conformation.

In particular embodiments of the invention, each protomer of said trimer recombinant SARS-CoV-2 S glycoprotein ectodomain is such that:
the amino acid residues situated at positions 682 to 685 in the amino acid sequence of the native SARS-CoV-2 S glycoprotein (SEQ ID No: 1) are substituted by an amino acid motif of sequence GSAS (SEQ ID No: 2); and/or
it is linked to a C-terminal trimerization domain; and/or
it comprises at least two proline substitutions at positions 986 and 987 of the amino acid sequence of the native SARS-CoV-2 S glycoprotein (SEQ ID No: 1); and/or
it is linked to at least one tag at its C-terminal end; and/or
it comprises, in particular it consists of, the 1208 first amino acid residues of the SARS-CoV-2 S glycoprotein or a protein having at least 90% amino acid sequence identity therewith.

In particular embodiments:
the amino acid residue located at position 408 in the amino acid sequence of the native SARS-CoV-2 S glycoprotein (SEQ ID No: 1) is an arginine residue, the amino acid residue located at position 378 in the amino acid sequence of the native SARS-CoV-2 S glycoprotein (SEQ ID No: 1) is a lysine residue, and said arginine residue of one of said protomers and said lysine residue of another one of said protomers are linked by a methylene bridge; and/or
the amino acid residue located at position 947 in the amino acid sequence of the native SARS-CoV-2 S glycoprotein (SEQ ID No: 1) is a lysine residue, the amino acid residue located at position 1019 in the amino acid sequence of the native SARS-CoV-2 S glycoprotein (SEQ ID No: 1) is an arginine residue, and said lysine residue of one of said protomers and said arginine residue of another one of said protomers are linked by a methylene bridge; and/or
the amino acid residue located at position 947 in the amino acid sequence of the native SARS-CoV-2 S glycoprotein (SEQ ID No: 1) is a lysine residue, the amino acid residue located at position 776 in the amino acid sequence of the native SARS-CoV-2 S glycoprotein (SEQ ID No: 1) is a lysine residue and said lysine residues are linked by a methylene bridge.

In particular embodiments of the invention, the trimer is a homomeric trimer.

According to the invention, a method of producing such a recombinant SARS-CoV-2 S glycoprotein ectodomain trimer comprises expressing nucleic acid molecule(s) encoding said protomers in a host cell to produce said trimer, purifying said trimer and treating said trimer with formaldehyde.

Another object of the invention is a proteoliposome comprising a lipid vesicle a surface of which is coated by a recombinant SARS-CoV-2 S glycoprotein ectodomain trimer of the invention.

A method of preparing such a proteoliposome comprises incubating said trimer with said lipid vesicle.

Another object of the invention is a vaccine comprising proteoliposomes of the invention, and optionally a pharmaceutically acceptable carrier and/or an adjuvant.

The invention also relates to a method of treating or preventing a SARS-CoV-2 infection in a subject, comprising administering to the subject a therapeutically effective amount of the vaccine of the invention.

In particular embodiments of the invention, the method comprises administering a therapeutically effective amount of the vaccine to the subject at least twice, or at least three times.

The vaccine may in particular be administered to the subject intramuscularly or intranasally.

### BRIEF DESCRIPTION OF THE DRAWINGS

The features and advantages of the invention will emerge more clearly in the light of the following examples of implementation, provided for illustrative purposes only and in no way limitative of the invention, with the support of FIGS. 1 to 7.

FIG. 1 shows the results of structural characterization of a trimer of the invention (FA-S) and proteoliposomes based on this trimer (FA-S-LVs). (A) Left panel, cryo-EM density of FA-S with all three RBD down. The structure was calculated from 126,719 particles imposing C3 symmetry; middle panel, molecular model of FA-S refined to a resolution of 3.4 Å shown as ribbon. Modeled N-linked glycans are shown as all atom models; right panel, two major cross-linking sites were identified that covalently link RBDs and the S2 subunits from different protomers. (B) Close-up of the cross-linking sites between RBDs (left panel); formaldehyde cross-linked amino groups of K378 and R408 of neighboring protomers as indicated by the continuous density connecting side chains (right panel). (C) Close-up of the cross-linking sites between S2 (left panel); continuous density between the central helix R1019 as well as S2 K776 to S2 HR1 K947 shows two alternative cross-links between protomers with equal occupancy (right panel). (D) Analyzis by negative staining electron microscopy of FA-S-LVs, revealing regular decoration of the liposomes with the S trimer. Counting FA-S trimer on 50 FA-S-LVs (negative staining EM two-dimensional vision) indicated 231±92 trimers. It is thus estimated that approximately or at least 460±184 FA-S trimers are attached to the LVs. Scale bar, 200 nm.

FIG. 2 shows the results of analysis of antibody responses induced by FA-S-LVs vaccination of cynomolgus macaques. (A) Scheme of vaccination, challenge and sampling. Syringes indicate the time points of vaccination and the virus particle indicates the time point of challenge. Symbols of identifying individual macaques are used in all figures. (B) ELISA of SARS-CoV-2 S-protein-specific IgG determined during the study at weeks 0, 2, 4, 6, 8, 10, 12, 22, 24, 26, 28, Ab titers of individual animals are shown. (C) ELISA of SARS-CoV-2 FA-S-protein-specific IgG determined during the study at the indicated weeks. (D) ELISA of SARS-CoV-2 S RBD-specific IgG determined during the study at the indicated weeks. For panels (B), (C), and (D), differences between matched groups were compared using the Wilcoxon signed-rank test (p<0.1). (E and F) Detection of S-specific IgG (E) and IgA (F) in nasopharyngeal fluids. Relative mean fluorescence intensity (MFI) of IgG and IgA binding to SARS-CoV-2 S measured with a Luminex-based serology assay in nasopharyngeal swabs. The background level is indicated by dotted lines. The vertical line indicates the day of challenge. For panels (E), and (F), groups were compared using the Mann-Whitney U test (*p<0.05). Data presented in A to F are from technical duplicates.

FIG. 3 shows the serum neutralization of SARS-CoV-2 pseudovirus upon FA-S-LVs vaccination. (A) The evolution of SARS-CoV-2 neutralizing Ab titers is shown for sera collected at weeks 0, 2, 4, 6, 8, 11, 12, 19. Bars indicate median titers of the four animals. Differences between matched groups were compared using the Wilcoxon signed-rank test (p<0.1). Data presented are from technical duplicates. (B) Serum from week 11 was depleted of RBD-specific Abs by affinity chromatography and neutralization activity of the complete serum of each animal was set to 100% and compared to the RBD-depleted sera and the RBD-specific sera.

FIG. 4 shows results of FA-S-LVs immunization of cynomolgus macaques against SARS-CoV-2 infection. Genomic (A) and subgenomic (sg)RNA viral loads (B) in tracheal swabs (left) and nasopharyngeal swabs (middle) of control (black) and vaccinated (grey) macaques after challenge. Viral loads in control and vaccinated macaques after challenge in BAL are shown (right). Bars indicate median viral loads. Vertical dotted lines indicate the day of challenge. Horizontal dotted lines indicate the limit of quantification. Data presented are from technical duplicates.

FIG. 5 shows the results of analysis of serum antibody titers and neutralization of vaccinated and control cynomolgus macaques after SARS CoV-2 challenge. Antibody IgG titers were determined by ELISA at weeks 24 (challenge), 25, 26, 27 and 28 against (A) SARS-CoV-2 S, (B) SARS-CoV-2 FA-S and (C) SARS-CoV-2 S RBD. Vaccinated animals are shown with grey symbols and control animals with black symbols. (D) SARS CoV-2 pseudovirus neutralization titers at week 24 (challenge) and 1, 2 and 4 weeks post exposure (weeks 25, 26, 28). The Bars show the median titers. For panels A to D, differences between matched groups were compared using the Wilcoxon signed-rank test (p<0.1). Data presented in A to D are from technical duplicates.

FIG. 6 illustrates the antigen-specific CD4 T-cell responses in FA-S-LV immunized cynomolgus macaques. Frequency of (A) IFNγ+, TFNα+ and IL-2+, (B) Th1 (IFN γ+/−, IL-2+/−, TNFα+), (C) IL-13+ and (D) IL-17+ antigen-specific CD4+ T cells (CD154+) in the total CD4+ T cell population, respectively, for each immunized macaque (n=4) at week (W)21 post-immunization (p.im.) (i.e. two weeks after the 4^{th} immunization, pre-exposure) and 14 days post-exposure (dpe.). PBMCs were stimulated overnight with culture medium only (“NS”, light grey symbols) or with SARS-CoV-2 S overlapping peptide pools (“S1+S2”, grey symbols). Bars indicate means. Time points in each experimental group were compared using the Wilcoxon signed rank test.

FIG. 7 shows robust neutralization of SARS CoV-2 variants induced by FA-S-LVs vaccination. B.1.1.7 (Alpha, UK), B.1.351 (Beta, SA) and P.1 (Gamma, BR) pseudovirus neutralization titers were compared to the Wuhan vaccine strain. Titers were determined using total IgG purified from sera at weeks 8 (2 immunizations), 12 (3 immunizations), 24 and 28 (4 immunizations). Background neutralization by IgG isolated from naïve animals was <100 for all variants and is indicated by the dashed line. Data presented are from technical duplicates.

### DESCRIPTION OF THE PREFERRED EMBODIMENTS

Recombinant SARS-CoV-2 S Glycoprotein Ectodomain Trimer

The recombinant SARS-CoV-2 S glycoprotein ectodomain trimer of the invention comprises three recombinant protomers each comprising at least the SARS-CoV-2 virus S glycoprotein ectodomain.

In particularly preferred embodiments of the invention, these three protomers are identical, the trimer of the invention then being a homomeric trimer.

In alternative embodiments of the invention, these three protomers are all different from one another, or two of them are identical and the third one is different.

The SARS-CoV-2 S glycoprotein ectodomain is herein defined, in a conventional way, as the domain of the protein that extends into the extracellular space. More particularly, in the amino acid sequence of the S glycoprotein of the firstly identified SARS-CoV-2 virus (Severe acute respiratory syndrome coronavirus 2 isolate Wuhan-Hu-1), the ectodomain of the SARS-CoV-2 S glycoprotein corresponds to amino residues 12 to 1190. The amino acid sequence of this S glycoprotein, herein denoted “native SARS-CoV-2 S glycoprotein” (SEQ ID No: 1) is described in the GenBank database under accession number MN908947.3 (“S” coding sequence (nucleotides 21563 to 25384)).

By SARS-CoV-2 S glycoprotein ectodomain, it is included according to the invention the ectodomain of the native SARS-CoV-2 S glycoprotein, of amino acid sequence SEQ ID No: 3, herein denoted “native ectodomain”, as well as any variant thereof retaining the capacity of the native ectodomain of inducing a neutralizing antibody response and preferably having an amino acid sequence with at least 90%, preferably at least 95%, preferably at least 98% and even more preferably at least 99%, identity with the amino acid sequence SEQ ID No: 3.

Such variants may consist of the S glycoprotein ectodomain of any variant or mutant of the firstly identified SARS-CoV-2 virus, such as the variant Alpha (also known as the variant B.1.1.7), the variant Beta (also known as the variant B.1.351), the variant Gamma (also known as the variant P.1), the variant Delta (also known as the variant B.1.617.2), the variant Omicron (also known as the variant B.1.1.529), etc., or they may consist of any protein having at least 90%, preferably at least 95%, preferably at least 98% and even more preferably at least 99%, amino acid identity therewith and retaining the capacity thereof of inducing a neutralizing antibody response.

Variants of the native SARS-CoV-2 S glycoprotein ectodomain may in particular have, relative to the sequence of the native ectodomain, which constitutes the reference sequence, insertions, deletions and/or substitutions, in particular N-terminal and/or C-terminal modifications, and/or non-native bonds between amino acid residues. In the case of a substitution, this is preferably carried out by an amino acid of the same family as the original amino acid, for example by substitution of a basic residue such as arginine by another basic residue such as a lysine residue, of an acid residue such as aspartate by another acid residue such as glutamate, of a polar residue such as serine by another polar residue such as threonine, of an aliphatic residue such as leucine by another aliphatic residue such as isoleucine, etc.

The percentage of identity between two amino acid sequences is herein determined in a conventional way in itself, by comparing the two optimally aligned sequences, through a comparison window. The amino acid sequence to be compared and located in the comparison window may include additions or deletions with respect to the reference sequence so as to obtain an optimal alignment between the two sequences. The percentage identity is then calculated by determining the number of positions for which an amino acid residue is identical in the two sequences compared, then dividing this number of positions by the total number of positions in the window of comparison, the number obtained being multiplied by one hundred to obtain the percentage of identity between the two sequences.

Variants of the native SARS-CoV-2 S glycoprotein ectodomain described in the prior art as having improved stability, in particular in a prefusion state, are particularly preferred in the context of the invention.

In particular embodiments, at least one, preferably two and more preferably the three, of the protomers of the invention consist of the SARS-CoV-2 S glycoprotein ectodomain. In alternative embodiments, it/they also comprise(s) additional amino acid residues of the SARS-CoV-2 S glycoprotein, for example residues 1 to 11 and/or residues 1191 to 1208 thereof.

In all this description, the amino acids positions are given in reference to the sequence of the native SARS-CoV-2 S glycoprotein (SEQ ID No: 1). These positions may be slightly different according to the variants of the SARS-CoV-2 S glycoprotein considered. It is within the skills of the person skilled in the art to identify these positions for a given variant, based on the particular amino acids and on a comparison of the variant's sequence with the native sequence. Moreover, these positions do not necessarily correspond to the positions in the actual recombinant protomers, if the latter do not comprise the first amino acids of the SARS-CoV-2 S glycoprotein situated upstream the ectodomain of this S glycoprotein. It is within the skills of the person skilled in the art to identify the position, in the protomer, of an amino acid residue defined in the context of the invention by its position in the native SARS-CoV-2 S glycoprotein sequence.

Each protomer of the trimer of the invention may comprise, or consist of, the 1208 first amino acid residues of the SARS-CoV-2 S glycoprotein, or a protein having at least 90%, preferably at least 95%, preferably at least 98%, and more preferably at least 99%, amino acid sequence identity therewith and retaining the capacity thereof to induce a neutralizing antibody response. Such a protein may in particular comprise one or more amino acid substitutions, insertions and/or deletions with respect to the 1208 first amino acid residues of the SARS-CoV-2 S glycoprotein.

In the recombinant SARS-CoV-2 S glycoprotein ectodomain trimer of the invention, in each protomer, the furin cleavage site is inactivated/disrupted. This furin cleavage site is formed of the four amino acid residues corresponding to the residues that are situated at positions 682 to 685 in the amino acid sequence of the native SARS-CoV-2 S glycoprotein (SEQ ID No: 1) (arg682, arg683, ala684 and arg685 in the amino acid sequence of the native SARS-CoV-2 S glycoprotein).

Such an inactivation of this furin cleavage site increases the cellular stability of the recombinant SARS-CoV-2 S glycoprotein ectodomain trimer, by preventing any cleavage between the S1 and S2 domains by cellular proteases such as furin.

In particular embodiments, at least one, preferably two, and more preferably each, protomer of the trimer of the invention comprises 682G, 683S, 684 Å and/or 685S substitution(s).

In particular embodiments, in at least one, preferably two, and more preferably in each, protomer of the trimer of the invention, the amino acid residues corresponding to those situated at positions 682 to 685 in the amino acid sequence of the native SARS-CoV-2 S glycoprotein (SEQ ID No: 1) are substituted by an amino acid motif of sequence GSAS (SEQ ID No: 2).

In the recombinant SARS-CoV-2 S glycoprotein ectodomain trimer of the invention, the amino acid residue corresponding to the amino acid residue at position 408 in the amino acid sequence of the native SARS-CoV-2 S glycoprotein (SEQ ID No: 1) of one of said protomers is covalently linked to the amino acid residue corresponding to the amino acid residue at position 378 in the amino acid sequence of the native SARS-CoV-2 S glycoprotein (SEQ ID No: 1) of another one of said protomers. This cross-linking between at least two protomers of the trimer advantageously keeps the latter in the native closed “RBD-down” conformation. These amino acids may be directly linked to each other by a covalent bond, or covalently linked by a spacer arm. As an example, these amino acids can be linked to each other by a methylene bridge. Otherwise, this cross-linking between the one protomer and the other protomer can be a non-native disulfide bond between cysteine residues introduced by suitable substitutions in the protomers.

Preferably, at least one protomer is covalently linked to another protomer by an intermolecular linkage between the amino acid at position 408 of the one protomer and the amino acid at position 378 of the other protomer. These amino acid residues can for example be attached to one another by a methylene bridge or by a non-native disulfide bond between cysteine residues introduced by a 378C substitution in the one protomer and a 408C substitution in the other protomer.

In particular embodiments of the invention, in at least one protomer, preferably in each protomer, the amino acid residue at position 408 of the amino acid sequence of the native SARS-CoV-2 S glycoprotein (SEQ ID No: 1) is an arginine residue (Arg 408), and in at least another protomer, preferably in each protomer, the amino acid residue at position 378 of the amino acid sequence of the native SARS-CoV-2 S glycoprotein (SEQ ID No: 1) is a lysine residue (Lys378), and said arginine residue of one of said protomers and said lysine residue of another one of said protomers are linked by a methylene bridge.

In the recombinant SARS-CoV-2 S glycoprotein ectodomain trimer of the invention, at least one protomer is covalently linked to another protomer by an intermolecular linkage between S2 subunits. More particularly, the amino acid residue corresponding to the amino acid residue at position 947 in the amino acid sequence of the native SARS-CoV-2 S glycoprotein (SEQ ID No: 1) of one of the protomers is covalently linked to the amino acid residue corresponding to the amino acid residue at position 1019 in the amino acid sequence of the native SARS-CoV-2 S glycoprotein (SEQ ID No: 1) of another one of the protomers, situated in the central S2 helix. This additional cross-linking between at least two protomers of the trimer advantageously stabilizes the trimer. These amino acid residues may be directly linked to each other by a covalent bond, or covalently linked by a spacer arm. As an example, these amino acid residues can be linked to each other by a methylene bridge.

Preferably, at least one protomer is covalently linked to another protomer by an intermolecular linkage between the amino acid residue at position 947 of the one protomer and the amino acid residue at position 1019 of the other protomer, in the central S2 helix. These amino acid residues can for example be linked to each other by a methylene bridge attached to a lysine residue at position 947 of the one protomer and to an arginine residue at position 1019 of the other protomer.

In particular embodiments of the invention, in at least one protomer, preferably in each protomer, the amino acid residue at position 947 of the amino acid sequence of the native SARS-CoV-2 S glycoprotein (SEQ ID No: 1) is a lysine residue (Lys947), and in at least another protomer, preferably in each protomer, the amino acid residue at position 1019 of the amino acid sequence of the native SARS-CoV-2 S glycoprotein (SEQ ID No: 1) is an arginine residue (Arg 1019), and said lysine residue of one of said protomers and said arginine residue of another one of said protomers are linked by a methylene bridge.

Alternatively, or additionally, in the recombinant SARS-CoV-2 S glycoprotein ectodomain trimer of the invention, at least one protomer is covalently linked to another protomer by a different intermolecular linkage between S2 subunits. More particularly, the amino acid residue corresponding to the amino acid residue at position 947 in the amino acid sequence of the native SARS-CoV-2 S glycoprotein (SEQ ID No: 1) of one of the protomers is covalently linked to the amino acid residue corresponding to the amino acid residue at position 776 in the amino acid sequence of the native SARS-CoV-2 S glycoprotein (SEQ ID No: 1) of another one of the protomers. This additional cross-linking between at least two protomers of the trimer also advantageously stabilizes the trimer. These amino acids may be directly linked to each other by a covalent bond, or covalently linked by a spacer arm. As an example, these amino acids can be linked to each other by a methylene bridge.

Preferably, at least one protomer is covalently linked to another protomer by an intermolecular linkage between the amino acid residue at position 947 of the one protomer and the amino acid at position 776 of the other protomer. These amino acid residues can for example be linked to each other by a methylene bridge attached to a lysine residue at position 947 of the one protomer and to a lysine residue at position 776 of the other protomer.

In particular embodiments of the invention, in at least one protomer, preferably in each protomer, the amino acid residue at position 947 of the amino acid sequence of the native SARS-CoV-2 S glycoprotein (SEQ ID No: 1) is a lysine residue (Lys947), and in at least another protomer, preferably in each protomer, the amino acid residue at position 776 of the amino acid sequence of the native SARS-CoV-2 S glycoprotein (SEQ ID No: 1) is a lysine residue (Lys776) and said lysine residue at position 947 of one of said protomers and said lysine residue at position 776 of another one of said protomers are linked by a methylene bridge.

The trimer of the invention can comprise any number and any combination of the intermolecular covalent linkages described above.

These covalent intermolecular linkages may for example be obtained by treating a recombinant SARS-CoV-2 S glycoprotein ectodomain trimer with formaldehyde.

The inventors have discovered that, surprisingly, these covalent intermolecular linkages stabilize the recombinant SARS-CoV-2 S glycoprotein ectodomain trimer, advantageously in the closed RBD-down conformation, over a long period, preventing conformational changes leading to the post-fusion conformation, while not preventing antibodies production and not masking the epitopes recognized by the antibodies. Therefore, the recombinant SARS-CoV-2 S glycoprotein ectodomain trimer of the invention enables the production of antibodies recognizing the tridimensional epitopes of the native protein.

The trimer of the invention can also comprise additional covalent linkages, in particular via methylene bridges, which may be intermolecular or intramolecular linkages. These additional linkages can be between the above-mentioned amino acid residues, between such residues and other amino acid residues, or between other amino acid residues.

In the recombinant SARS-CoV-2 S glycoprotein ectodomain trimer of the invention, one, two or the three protomers can comprise C-terminal and/or N-terminal modification(s).

In particular embodiments of the invention, at least one, preferably two, and more preferably each, of the protomers is linked to a C-terminal trimerization domain, which increases its stability in the trimer form, for example a C-terminal T4 fibritin trimerization domain, or any other domain known by the person skilled in the art for its capacity of triggering and/or stabilizing trimerization of a protein to which it is linked.

In preferred embodiments of the invention, each protomer comprises, at its C-terminal end, a C-terminal T4 fibritin trimerization motif, also known as Foldon domain, of amino acid sequence GYIPEAPRDGQAYVRKDGEWVLLSTFL (SEQ ID No: 4), consisting of an extended N-terminal region (G1-Q11), a β-hairpin (A12-L23), and a C-terminal 3₁₀ helix (L23-L27). The extended N-terminal region contains a polyproline II helix between residues P4 and P7 and packs against one side of the β-hairpin by hydrophobic contacts.

In particular embodiments of the invention, at least one, preferably two, and more preferably each, of the protomers comprises at least two proline substitutions at amino acid residues corresponding to the amino acid residues at positions 986 and 987 in the amino acid sequence of the native SARS-CoV-2 S glycoprotein (SEQ ID No: 1). These proline substitutions increase the stability of the trimer in its native conformation in the cells.

In particular embodiments of the invention, at least one, preferably two, and more preferably each, of the protomers comprises two to four additional proline substitutions, preferably at amino acid residues corresponding to the amino acid residues at positions 817, 892, 899 and/or 942 in the amino acid sequence of the native SARS-CoV-2 S glycoprotein (SEQ ID No: 1). These additional proline substitutions increase even more the stability of the trimer in its native conformation in the cells.

In particular embodiments of the invention, at least one, preferably two, and more preferably each, of the protomers is linked, preferably at its C-terminal end, to at least one tag, such as a twin Strep-Tag® and/or a polyhistidine tag (for example of eight successive histidine residues), for facilitating its purification.

The twin Strep-Tag® consists of two spaced apart identical amino acid sequences of sequence WSHPQFEK (SEQ ID No: 5) and has the capacity of specifically binding to a specifically engineered streptavidin.

Each protomer may further comprise, between the C-terminal trimerization motif and the one or several tags, a cleavage site such as a HRV3C protease cleavage site, of sequence LEVLFQGP (SEQ ID No: 6).

A recombinant SARS-CoV-2 S glycoprotein ectodomain trimer according to the invention can comprise at least one, preferably two, and more preferably three, protomers comprising the amino acid sequence of sequence SEQ ID No: 7.

In particular embodiments, at least one, preferably two, and more preferably each, of the protomers consists of an amino acid sequence of sequence SEQ ID No: 8.

Method of producing the recombinant SARS-CoV-2 S glycoprotein ectodomain trimer A method of producing a trimer according to the invention comprises:
expressing nucleic acid molecule(s) encoding the recombinant protomer(s) of the invention, as defined above, in a host cell to produce a trimer of such protomer(s),
purifying this trimer, and
treating this trimer with formaldehyde.

The first step of this method is the production of the three protomers containing at least the SARS-CoV-2 S glycoprotein ectodomain by genetic recombination.

This step can be carried out by any manner known to the person skilled in the art, in particular using an expression vector comprising nucleic acid molecules encoding these protomers. When all the protomers are identical, only one such nucleic acid molecule is required.

The expression vector can be of any type known per se for use in genetic engineering, in particular a plasmid, a cosmid, a virus, a bacteriophage, containing the necessary elements for the transcription and translation of the sequence(s) encoding the protomer(s) according to the invention. It can comprise in particular the following elements, functionally linked, for each protomer: a promoter located 5′ of a nucleotide sequence coding for the protomer according to the invention, and transcription termination signals 3′ of this sequence.

The host cell can be any cell in which a nucleic acid molecule can be expressed.

This host cell can equally well be a prokaryotic cell, in particular bacterial, particularly for the mass production of the protomer(s), or a eukaryotic cell, which can be of lower or higher eucaryote, for example of yeast, invertebrates or mammals. The host cell may express the protomer(s) of the invention in a stable, inducible or constitutive manner, or else in a transient manner. As an example, the nucleic acid molecule(s) encoding the protomer(s) of the invention can be transiently expressed in human embryonic kidney cell lines.

The host cell is cultured under conditions enabling the expression and the recovery of the protomer(s) thus produced, which are naturally assembled in a trimer form. It is within the skills of the person skilled in the art to determine such culture conditions, according to the cell type.

The trimer thus obtained may be purified by any method known per se. In the particular embodiments of the invention wherein at least one of the recombinant protomers comprises a tag, in particular at its C-terminal end, such as a poly-histidine tag, the purification method can take advantage of the specific capacity of this tag to bind a binding partner, for example Sepharose resin in the case of the poly-histidine tag.

The step of treating the trimer with formaldehyde is carried out in conditions enabling the formation of intramolecular methylene bridges between the protomers, for example:
between an amino acid residue at position 408, preferably Arg408, and an amino acid residue at position 378, preferably Lys378,
and between an amino acid residue at position 947, preferably Lys947, and an amino acid residue at position 1019, preferably Arg1019, and/or between an amino acid residue at position 947, preferably Lys947, and an amino acid residue at position 776, preferably Lys 776.

It is preferably carried out for several hours, at least for 2 hours, and preferably overnight in order to obtain a complete crosslinking between the protomers. It can be carried out at room temperature, i.e., at a temperature of between 20 and 25° C.

As an example, the step of treating the trimer with formaldehyde comprises contacting the trimer with an aqueous solution comprising 4% v/v of formaldehyde. The content of protein introduced in this aqueous solution is preferably comprised between 0.3 and 1.2 mg/ml. It is for example of about 1 mg/ml.

Proteoliposomes

A proteoliposome according to the invention comprises a lipid vesicle a surface of which is coated by a recombinant SARS-CoV-2 S glycoprotein ectodomain trimer according to the invention.

Such a proteoliposome, preferably of defined lipid composition, advantageously resembles a virus-like particle and provides increased stability and prolonged circulating half-life in vivo of the recombinant trimer. Used as a vaccine, the proteoliposome of the invention induces more efficient immunes responses than immunization with the sole recombinant trimer of the invention.

The lipid vesicle may be of any type known per se for an administration to a living being, for example a human being, in particular as a vaccine.

In particular embodiments of the invention, the lipid vesicle comprises a mixture of L-α-phosphatidylcholine, cholesterol and a polyhistidine-tag conjugating lipid, in particular 56 to 61% by weight of L-α-phosphatidylcholine, 34 to 36% by weight of cholesterol and 3 to 10% by weight of a polyhistidine-tag conjugating lipid, for example 60% by weight of L-α-phosphatidylcholine, 36% by weight of cholesterol and 4% by weight of a polyhistidine-tag conjugating lipid.

The polyhistidine-tag conjugating lipid can for example be a lipid modified by chelator immobilizing a metal cation, such as a nickel or a cobalt cation. The product marketed by Avanti® Polar Lipids under the name DGS-NTA-(Ni²⁺) (1,2-dioleoyl-sn-glycero-3-[(N-(5-amino-1-carboxypentyl)iminodiacetic acid)succinyl](nickel salt)) can for example be used.

Alternatively, cobalt porphyrin phospholipids (CoPoP) incorporated into lipid bilayers can be used to attach polyhistidine-tagged glycoproteins (Federizon et al, 2021, Pharmaceutics, 13, 98), more particularly, in the context of the invention, the recombinant SARS-CoV-2 S glycoprotein ectodomain trimer of the invention.

Method of Preparing the Proteoliposomes

A method of preparing proteoliposomes according to the invention comprises incubating the recombinant SARS-CoV-2 S glycoprotein ectodomain trimer of the invention with the lipid vesicle, so as to ensure immobilization of the trimer on the surface of the lipid vesicle by non-covalent interactions.

This incubation is preferably carried out for at least 6 hours, preferably from 12 to 24 hours, and preferably at a temperature of between 20 and 25° C.

The ratio trimer/lipid vesicle is preferably chosen so as to have a sufficient excess of protein to saturate the lipid vesicles, more particularly at least 2 times of excess protein. The ratio trimer/lipid vesicle is preferably comprised between 2:1 w/w and 4:1 w/w. It is for example equal to 3:1 w:w.

Alternatively, the recombinant trimer of the invention can be covalently linked to the surface of the lipid vesicle, for example by reacting at least a cysteine residue of at least a protomer of the trimer, preferably a cysteine residue situated in a C-terminal part of the protomer, with a group carried by the lipid vesicle, capable of reacting with a sulfhydryl group.

The method may comprise a final step of purifying the proteoliposome obtained.

Vaccine

A vaccine according to the invention comprises proteoliposomes such as defined above, and, optionally, a pharmaceutically acceptable carrier and/or an adjuvant.

The pharmaceutically acceptable carrier can consist of any conventional carrier, in particular in the field of vaccine compositions. It is for example a liquid aqueous carrier.

Any adjuvant capable of enhancing an immune response of the host can be contained in the vaccine of the invention. Examples of adjuvants are based on monophosphoryl lipid A (MPLA) and aluminum salt, for example aluminum hydroxide or aluminum phosphate, such as AS504, or on squalene, such as MF59.

The vaccine according to the invention can furthermore contain any conventional additive known per se, as well as optionally other active substances.

As additives that can be used in the vaccine according to the invention, mention can be made of excipients, diluents, surfactants, in particular of polysorbate type, stabilizing agents, etc.

The vaccine according to the invention can be formulated in any dosage form suitable for administration to a mammal, in particular to a human subject. It is preferably formulated in a dosage form suitable for parenteral administration. In particular, it can be formulated in a dosage form suitable for intramuscular, intravenous, intraperitoneal or subcutaneous injection, or for administration by the intranasal route or by inhalation. It is for example formulated as a solution for injection or as a formulation for spray.

The vaccine of the invention can be conditioned in monodose form or in multidose form, for example in a five-dose vial. In particular embodiments of the invention, a unit dose of the vaccine comprises 50 to 100 μg of proteoliposomes of the invention.

Method of Treating or Preventing a SARS-CoV-2 Infection in a Subject

A method of treating or preventing a SARS-CoV-2 infection in a subject comprises administering to this subject a therapeutically effective amount of the vaccine of the invention, so as to induce an immune response to the SARS-CoV-2 glycoprotein S ectodomain.

The subject is preferably a mammal, in particular a human subject.

The vaccine of the invention can be used to treat any subject in need thereof, in particular any subject suffering from a SARS-CoV-2 infection, or, by way of prevention, any non-affected subject likely to contract such an infection.

In particular, it has been discovered by the inventors that immunization of cynomolgus macaques with the vaccine of the invention induces high antibody titers with potent neutralizing activity against the vaccine strain, alpha, beta and gamma variants as well as TH1 CD4+ biased T cell responses. High titers are already induced after two immunizations, with a median ID50 of about 8000 two weeks after the second immunization. Furthermore, although anti-RBD specific antibody responses are initially predominant, the third immunization boosts significant non-RBD antibody titers. Four weeks after the third immunization, median S-specific ED50s are 3 times higher than RBD-specific ED50s. This trend is continued after the fourth immunization which reveals a 3.5 times higher median ID50 for S than for RBD five weeks post immunization. These results show that more than two immunizations allow to expand the reactive B cell repertoire that target non-RBD S epitopes. Furthermore, challenging of vaccinated animals with SARS-CoV-2 shows complete protection through sterilizing immunity. In particular, no signs of virus replication can be detected in the upper and lower respiratory tracts consistent with the absence of clinical signs of infection such as lymphopenia and lung damage characteristics for Covid-19 disease. Significant IgG and IgA are detected in nasopharyngeal fluids at the time of viral challenge, showing that administration of the vaccine of the invention induces sterilizing protection by eliciting mucosal immune responses. Therefore, the vaccine of the invention is efficient and safe.

This administration of the vaccine of the invention can be carried out by any route. It is preferably carried out by a parenteral route, in particular by injection or spray.

In particularly preferred embodiments of the invention, the vaccine is administrated to the subject intramuscularly or intranasally.

Alternatively, it can be administered to the subject by intravenous, intraperitoneal, intraarterial or subcutaneous injection, or by inhalation.

The vaccine of the invention can be administered to the subject in a single dose, or in several doses, in particular administered several days apart.

The therapeutically effective dose and the number of administered doses are dependent on the subject, in particular on its age, weight, symptoms, etc.

Determining the exact administration conditions is within the remit of the practitioner.

For example, each administration is carried out to deliver between 50 and 100 μg of proteoliposomes of the invention to the subject.

Preferably, the method of the invention comprises administering a therapeutically effective amount of the vaccine at least twice to the subject. The two administration steps are preferably spaced apart by a period of between 2 and 8 weeks.

In particular embodiments of the invention, the method comprises administering a therapeutically effective amount of the vaccine at least three times to the subject. As indicated above, a third immunization advantageously boosts significant non-RBD antibody titers.

Alternatively, the vaccine of the invention can be administrated to a subject after two administrations of other anti-SARS-CoV-2 vaccine(s).

An aspect of the invention relates to the use of a vaccine according to the invention for treating or preventing a SARS-CoV-2 infection in a subject. This use comprises administering to the subject a therapeutically effective amount of the vaccine. It may respond to any of the features described above in relation with the method of the invention for treating or preventing a SARS-CoV-2 infection in a subject.

### EXAMPLES

Experimental Model and Subject Details

Cell Lines

HEK293T (ATCC CRL-11268) and HEK293F (Thermo Fisher Scientific) are human embryonic kidney cell lines. HEK293F cells are adapted to grow in suspension. HEK293F cells were cultured at 37° C. with 8% C02 and shaking at 125 rpm in 293FreeStyle expression medium (Life Technologies). HEK293T cells were cultured at 37° C. with 5% C02 in flasks with DMEM supplemented with 10% fetal bovine serum (FBS), streptomycin (100 μg/mL) and penicillin (100 U/mL). HEK293T/ACE2 cells are a human embryonic kidney cell line expressing human angiotensin-converting enzyme 2. HEK293T/ACE2 cells were cultured at 37° C. with 5% C02 in flasks with DMEM supplemented with 10% FBS, streptomycin (100 μg/mL) and penicillin (100 U/mL). VeroE6 cells (ATCC CRL-1586) are a kidney epithelial cells from African green monkeys. VeroE6 cells were cultured at 37° C. with 5% C02 in DMEM supplemented with or without streptomycin (100 μg/mL) and penicillin (100 U/mL) and with or without 5 or 10% FBS, and with or without TPCK-trypsin. PBMC were isolated from macaque sera and cultured in RPMI1640 GlutaMAX® medium (Gibco®) supplemented with 10% FBS.

Viruses

SARS-CoV-2 virus (hCoV-19/France/IDF0372/2020 strain) was isolated by the National Reference Center for Respiratory Viruses (Institut Pasteur, Paris, France) as described in Lescure etaL. (2020). Lancet Infect Dis 20, 697-706, and produced by two passages on Vero E6 cells in DMEM (Dulbecco's Modified Eagles Medium) without FBS, supplemented with 1% P/S (penicillin at 10,000 U·ml⁻¹ and streptomycin at 10,000 μg·ml-1) and 1 μg·ml⁻¹ TPCK-trypsin at 37° C. in a humidified CO₂ incubator and titrated on Vero E6 cells. Whole genome sequencing was performed with no modifications observed compared with the initial specimen and sequences were deposited after assembly on the GISAID EpiCoV platform under accession number ID EPI_ISL_410720.

Ethics and Biosafety Statement

Cynomolgus macaques (Macaca fascicularis) originating from Mauritian AAALAC certified breeding centers, described in Table 1, were used in this study. MF1-MF4 are in the vaccinated group and MF5-MF8 in the control group.

TABLE 1

| | | | | Weight at Day | |
|---|---|---|---|---|---|
| | | | Age | 0 post exposure | Developmental |
| Name | Gender | Date of birth | (years) | (kg) | stage |
| MF1 | M | 4 Apr. 2017 | 3.68 | 3.96 | Young adult |
| MF2 | M | 5 Apr. 2017 | 3.68 | 4.52 | Young adult |
| MF3 | M | 10 Apr. 2017 | 3.67 | 4.98 | Young adult |
| MF4 | M | 12 Apr. 2017 | 3.66 | 6.39 | Young adult |
| MF5 | M | 27 Apr. 2017 | 3.62 | 3.64 | Young adult |
| MF6 | M | 27 Apr. 2017 | 3.62 | 4.29 | Young adult |
| MF7 | M | 12 May 2017 | 3.58 | 3.14 | Young adult |
| MF8 | M | 15 May 2017 | 3.57 | 3.91 | Young adult |

All animals were housed in IDMIT infrastructure facilities (CEA, Fontenay-aux-roses), under BSL-2 and BSL-3 containment when necessary (Animal facility authorization #D92-032-02, Prefecture des Hauts de Seine, France) and in compliance with European Directive 2010/63/EU, the French regulations and the Standards for Human Care and Use of Laboratory Animals, of the Office for Laboratory Animal Welfare (OLAW, assurance number #A5826-01, US). The protocols were approved by the institutional ethical committee “Comité d'Ethique en Expérimentation Animale du Commissariat à l'Energie Atomique et aux Energies Alternatives” (CEtEA #44) under statement number A20-011. The study was authorized by the “Research, Innovation and Education Ministry” under registration number APAFIS #24434-2020030216532863.

Animals and Study Design

Cynomolgus macaques were randomly assigned in two experimental groups. The vaccinated group (n=4) received 50 μg of proteoliposomes of the invention (SARSCoV-2 S-LV) adjuvanted with 500 μg of MPLA liposomes (Polymun Scientific) diluted in PBS at weeks 0, 4, 8 and 19, while control animals (n=4) received no vaccination. Vaccinated animals were sampled in blood at weeks 0, 2, 4, 6, 8, 11, 12, 14, 19, 21 and 22. At week 24, all animals were exposed to a total dose of 10⁵ pfu of SARS-CoV-2 virus (hCoV-19/France/IDF0372/2020 strain; GISAID EpiCoV platform under accession number EPI_ISL_410720) via the combination of intranasal and intra-tracheal routes (0.25 mL in each nostril and 4.5 mL in the trachea, i.e., a total of 5 mL; day 0), using atropine (0.04 mg/kg) for pre-medication and ketamine (5 mg/kg) with medetomidine (0.042 mg/kg) for anesthesia. Nasopharyngeal, tracheal and rectal swabs, were collected at days 2, 3, 4, 6, 7, 10, 14 and 27 days post exposure (dpe) while blood was taken at days 2, 4, 7, 10, 14 and 27 dpe. Bronchoalveolar lavages (BAL) were performed using 50 mL sterile saline on 3 and 7 dpe. Chest CT was performed at 3, 7, 10 and 14 dpe in anesthetized animals using tiletamine (4 mg·kg⁻¹) and zolazepam (4 mg·kg⁻¹). Blood cell counts, haemoglobin, and haematocrit, were determined from EDTA blood using a DHX800 analyzer (Beckman Coulter).

Methods Details

Protein Expression and Purification

The gene of nucleotide sequence SEQ ID No: 9, encoding for a protein/protomer of amino acid sequence SEQ ID No: 8, corresponding to residues 1-1208 of the native SARS-CoV-2 S glycoprotein with proline substitutions at residues 986 and 987 (“2P”), a “GSAS” (SEQ ID No: 2) substitution at the furin cleavage site (residues 682-685), and linked at its C-terminal end to, successively: a T4 fibritin trimerization motif (of sequence SEQ ID No: 4), a C-terminal HRV3C protease cleavage site (of sequence SEQ ID No: 6), a 8X-histidine tag and a Twin-Strep-Tag® (two spaced-apart repeats of the sequence SEQ ID No: 5), was transiently expressed in human embryonic kidney cell lines FreeStyle293F (Thermo Fisher scientific) using polyethylenimine (PEI) 1 μg/μl for transfection.

Supernatants were harvested five days post-transfection, centrifuged for 30 min at 5000 rpm and filtered using 0.20 m filters (ClearLine®). The trimer (“S”) was purified from the supernatant by Ni²⁺-Sepharose chromatography (Excel purification resin, Cytiva) in buffer A (50 mM HEPES pH 7.4, 200 mM NaCl) and eluted in buffer B (50 mM HEPES pH 7.4, 200 mM NaCl, 500 mM imidazole). Eluted trimer-containing fractions were concentrated using Amicon® Ultra (cut-off: 30 KDa) (Millipore) and further purified by size-exclusion chromatography (SEC) on a Superose® 6 column (GE Healthcare) in buffer A or in PBS.

For RBD expression, the following reagent was produced under HHSN272201400008C and obtained through BEI Resources, NIAID, NIH: Vector pCAGGS containing the SARS-Related Coronavirus 2, Wuhan-Hu-1 Spike Glycoprotein Receptor Binding Domain (RBD), NR-52309. The SARS-CoV-2 S RBD domain (residues 319 to 541) was expressed in EXP1293 cells by transient transfection according to the manufacturer's protocol (Thermo Fisher Scientific). Supernatants were harvested five days after transfection and cleared by centrifugation. The supernatant was passed through a 0.45 μm filter and RBD was purified using Ni²⁺-chromatography (HisTrap HP column, GE Healthcare) in buffer C (20 mM Tris pH 7.5 and 150 mM NaCl buffer) followed by a washing step with buffer D (20 mM Tris pH 7.5 and 150 mM NaCl buffer, 75 mM imidazole) and elution with buffer E (20 mM Tris pH 7.5 and 150 mM NaCl buffer, 500 mM imidazole). Eluted RBD was further purified by SEC on a Superdex 75 column (GE Healthcare) in buffer C. Protein concentrations were determined using an absorption coefficient (A1%, 1 cm) at 280 nm of 10.4 and 13.06 for S protein and RBD, respectively, using ProtParam.

Trimer Crosslinking

The trimer “S protein” at 1 mg/ml in PBS was cross-linked with 4% formaldehyde (FA) (Sigma) overnight at room temperature. The reaction was stopped with 1 M Tris HCl pH 7.4 adjusting the sample buffer to 7.5 mM Tris/HCl pH 7.4. FA was removed by PBS buffer exchange using 30 KDa cut-off concentrators (Amicon®). FA crosslinking was confirmed by separating the formaldehyde-treated trimers “FA-S protein” on a 10% SDS-PAGE under reducing conditions.

Trimer Coupling to Liposomes

Liposomes were prepared as follows.

The liposomes were composed of 60% of L-α-phosphatidylcholine, 4% His tag-conjugating lipid, DGS-NTA-(Ni²⁺) and 36% cholesterol (Avanti Polar Lipids).

Lipid components were dissolved in chloroform, mixed and placed for two hours in a desiccator under vacuum at room temperature to obtain a lipid film. The film was hydrated in filtered (0.22 μm) PBS and liposomes were prepared by extrusion using membrane filters with a pore size of 0.1 μm (Whatman® Nuclepore® Track-Etched membranes). The integrity and size of the liposomes was analyzed by negative staining-EM.

For protein coupling, the liposomes were incubated overnight with FA-S protein or S protein in a 3:1 ratio (w/w).

Free FA-S protein was separated from the FA-S-proteoliposomes (S-LVs) by sucrose gradient (5-40%) centrifugation in a SW55 rotor at 40,000 rpm for 2 h.

The amount of protein conjugated to the liposomes was determined by Bradford assay and SDS-PAGE densitometry analysis comparing S-LV bands with standard S protein concentrations.

Trimer Thermostability

Thermal denaturation of S protein, native or FA-cross-linked was analyzed by differential scanning fluorimetry coupled to back scattering using a Prometheus NT.48 instrument (Nanotemper Technologies). Protein samples were first extensively dialyzed against PBS pH 7.4, and the protein concentration was adjusted to 0.3 mg/ml. 10 μL of sample were loaded into the capillary and intrinsic fluorescence was measured at a ramp rate of 1° C./min with an excitation power of 30%. Protein unfolding was monitored by the changes in fluorescence emission at 350 and 330 nm. The thermal unfolding midpoint (Tm) of the proteins was determined using the Prometheus NT software.

Negative Stain Electron Microscopy

Protein samples were visualized by negative-stain electron microscopy (EM) using 3-4 μL aliquots containing 0.1-0.2 mg/ml of protein. Samples were applied for 10 s onto a mica carbon film and transferred to 400-mesh Cu grids that had been glow discharged at 20 mA for 30 s and then negatively stained with 2% (wt/vol) Uranyl Acetate (UAc) for 30 s. Data were collected on a FEI Tecnai T12 LaB6-EM operating at 120 kV accelerating voltage at 23 k magnification (pixel size of 2.8 Å) using a Gatan Orius 1000 CCD Camera. Two-dimensional (2D) class averaging was performed with the software Relion using on average 30-40 micrographs per sample. The 5 best obtained classes were calculated from around 6000 particles each.

Cryo-Electron Microscopy

Data collection. 3.5 μL of sample were applied to 1.2/1.3 C-Flat® (Protochips Inc) holey carbon grids and plunged frozen in liquid ethane with a Vitrobot Mark IV (Thermo Fisher Scientific) (6 s blot time, blot force 0). The sample was observed with a Glacios® electron microscope (Thermo Fischer Scientific) at 200 kV. Images were recorded automatically on a K2 summit direct electron detector (Gatan Inc., USA) in counting mode with SerialEM. Movies were recorded for a total exposure of 4.5 s with 40 frames per movie and a total dose of 40 e−/Å². The magnification was 36,000× (1.15 Å/pixel at the camera level). The defocus of the images was changed between −1.0 and −2.5 μm. Two different datasets have been acquired on the same grid. First, 1040 movies were recorded with stage movement between each hole and then 7518 more movies were recorded with image shifts on a 3×3 hole pattern.

3D reconstruction. The movies were first drift-corrected with MotionCor2. The remaining image processing was performed with RELION 3.1.2 and CTF estimation with GCTF. An initial set of particles (box size of 200 pixels, sampling of 2.3 Å/pixel) was obtained by auto-picking with a Gaussian blob. After 2D classification, the best looking 2D class averages were used for a second round of auto picking. Following another 2D classification step, the particles belonging to the best looking 2D class averages were used to create an ab-initio starting 3D model which was then used to calculate a first 3D reconstruction with C3 symmetry. The 2D projections from that 3D model were then used to do one last auto picking which resulted in a total of 2,582,857 particles. Following another 2D classification and a 3D classification (Cl symmetry, 5 classes) steps, a 3D map at 4.6 Å resolution was obtained from 240,777 particles. The particles were re-extracted (box size of 400 pixels, sampling of 1.15 Å/pixel). After another 3D refinement (C3 symmetry) and 3D classification (Cl symmetry, no alignment, 3 classes) steps, a final set of 126,719 particles was identified which resulted in a 3D reconstruction at 3.6 Å resolution. Refinement of CTF parameters, particle polishing and a second round of CTF parameter's refinement further improved the resolution to 3.4 Å. The resolution was determined by Fourier Shell Correlation (FSC) at 0.143 between two independent 3D maps. The local resolution was calculated with blocres and found to be between 3 and 5 Å. The final 3D map was sharpened with DeepEMhancer.

Model refinement. The atomic model of the S protein in the closed conformation (PDB 6VXX) was rigid-body fitted inside the cryo-EM density map in CHIMERA. The atomic coordinates were then refined with PHENIX. The refined atomic models were visually checked and adjusted (if necessary) in COOT. The final model was validated with MOLPROBITY.

The figures were prepared with CHIMERA and CHIMERAX. The atomic coordinates and the cryo-EM map have been deposited in the Protein Data Bank and in the Electron Microscopy Data Bank under the accession codes 7QIZ and EMD-13776, respectively.

Virus Quantification in NHP Samples

Upper respiratory (nasopharyngeal and tracheal) and rectal specimens were collected with swabs (Viral Transport Medium, CDC, DSR-052-01). Tracheal swabs were performed by insertion of the swab above the tip of the epiglottis into the upper trachea at approximately 1.5 cm of the epiglottis. All specimens were stored between 2° C. and 8° C. until analysis by RT-qPCR with a plasmid standard concentration range containing an RdRp gene fragment including the RdRp-IP4 RT-PCR target sequence. SARS-CoV-2 E gene subgenomic mRNA (sgRNA) levels were assessed by RT-qPCR using the following primers and probe:
leader-specific primer sgLeadSARSCoV2-F of sequence:

(SEQ ID No: 10)
CGATCTCTTGTAGATCTGTTCTC,

E-Sarbeco-R primer of sequence:

(SEQ ID No: 11)
ATATTGCAGCAGTACGCACACA,

and E-Sarbeco probe of sequence:

(SEQ ID No: 12)
HEX-ACACTAGCCATCCTTACTGCGCTTCG-BHQ1,

wherein HEX represents hexachlorofluorescein and BHQ1 represents the BHQ1 quencher.

The protocol describing this procedure for the detection of SARS-CoV-2 is available on the WHO website (https://www.who.int/docs/default-source/coronaviruse/whoinhouseassays.pdf).

Chest CT and Image Analysis

Lung images were acquired using a computed tomography (CT) system (Vereos-Ingenuity, Philips), and analyzed using INTELLISPACE PORTAL 8 software (Philips Healthcare). All images had the same window level of −300 and window width of 1,600. Lesions were defined as ground glass opacity, crazy-paving pattern, consolidation or pleural thickening. Lesions and scoring were assessed in each lung lobe blindly and independently by two persons and the final results were established by consensus. Overall CT scores include the lesion type (scored from 0 to 3) and lesion volume (scored from 0 to 4) summed for each lobe.

ELISA

Serum antibody titers specific for soluble native S glycoprotein, FA-cross-linked trimer (FA-S) and for RBD were determined using an enzyme-linked immunosorbent assay (ELISA). Briefly, 96-well micro titer plates were coated with 1 μg of S, FA-S or RBD proteins at 4° C. overnight in PBS and blocked with 3% BSA for 1 h at room temperature after 3 washes with 150 μL PBS Tween®-20 0.05%. Serum dilutions were added to each well for 2 h at 37° C. and plates were washed 5 times with PBS Tween®. A horseradish peroxidase (HRP) conjugated goat anti-monkey H+L antibody (Invitrogen) was then added and incubated for 1 h before excess Ab was washed out and HRP substrate added. Absorbance was determined at 450 nm. Antibody titers were expressed as ED50 (effective Dilution 50-values) and were determined as the serum dilution at which IgG binding was reduced by 50%. ED50 were calculated from crude data (O.D) after normalization using GraphPad Prism (version 6) “log(inhibitor) vs normalized response” function. ELISA were performed in duplicates.

Protein Coupling to Luminex Beads

Proteins were covalently coupled to MagPlex® beads (Luminex Corporation) via a two-step carbodiimide reaction using a ratio of 75 μg S trimer to 12.5 million beads. MagPlex® beads were washed with 100 mM mono-basic sodium phosphate pH 6.2 and activated for 30 min on a rotor at RT by addition of Sulfo-N-Hydroxysulfosuccinimide (Thermo Fisher Scientific) and 1-Ethyl-3-(3-dimethylaminopropyl) carbodiimide (Thermo Fisher Scientific). The activated beads were washed three times with 50 mM MES pH 5.0 and added to S trimer, which was diluted in 50 mM MES pH 5.0. The coupling reaction was incubated for 3 h on a rotator at room temperature. The beads were subsequently washed with PBS and blocked with PBS containing 2% BSA, 3% FCS and 0.02% Tween®-20 for 30 min on a rotator at room temperature. Finally, the beads were washed and stored in PBS containing 0.05% sodium azide at 4° C. and used within 3 months.

Luminex Assay

50 μL of a working bead mixture containing 20 beads per μL was incubated overnight at 4° C. with 50 μL of diluted nasopharyngeal fluid. Nasopharyngeal fluids were diluted 1:20 for detection of S-specific IgG and IgA. Plates were sealed and incubated on a plate shaker overnight at 4° C. Plates were washed with TBS containing 0.05% Tween®-20 (TBST) using a hand-held magnetic separator. Beads were resuspended in 50 μL of Goat-anti-monkey IgG-Biotin or Goat-anti monkey IgA-Biotin (Sigma Aldrich) and incubated on a plate shaker at RT for 2 h. Afterwards, the beads were washed with TBST, resuspended in 50 μL of Streptavidin-PE (ThermoFisher Scientific) and incubated on a plate shaker at room temperature for 1 h. Finally, the beads were washed with TBST and resuspended in 70 μL Magpix® drive fluid (Luminex Corporation). The beads were agitated for a few minutes on a plate shaker at room temperature and then readout was performed on the MAGPIX® (Luminex Corporation). Reproducibility of the results was confirmed by performing replicate runs.

Pseudovirus Neutralization Assay

Pseudovirus was produced by co-transfecting the pCR3 SARS-CoV-2-SA19 expression plasmid (Wuhan Hu-1; GenBank: MN908947.3) with the pHIV-1 NL43 ΔEnv-NanoLuc reporter virus plasmid in HEK293T cells (ATCC, CRL-11268). The pCR3 SARS-CoV-2-SA19 expression plasmid contained the following mutations compared to the wild-type for the variants of concern: deletion (A) of H69, V70 and Y144, N501Y, A570D, D614G, P681H, T7161, S982 Å and D1118H in B.1.1.7 (Alpha, UK); L18F, D80A, D215G, L242H, R2461, K417N, E484K, N501Y, D614G and A701V in B.1.351 (Beta, SA); L18F, T20N, P26S, D138Y, R190S, K417T, E484K, N501Y, D614G, H655Y and T10271 in P.1 (Gamma, BR).

HEK293T/ACE2 cells were seeded at a density of 20,000 cells/well in a 96-well plate coated with 50 μg/mL poly-L-lysine 1 day prior to the start of the neutralization assay. Heat-inactivated sera (1:100 dilution) were serial diluted in 3-fold steps in cell culture medium (DMEM (Gibco), supplemented with 10% FBS, penicillin (100 U/mL), streptomycin (100 μg/mL) and GlutaMax® (Gibco), mixed in a 1:1 ratio with pseudovirus and incubated for 1 h at 37° C. These mixtures were then added to the cells in a 1:1 ratio and incubated for 48 h at 37° C., followed by a PBS wash and lysis buffer added. The luciferase activity in cell lysates was measured using the Nano-Glo® Luciferase Assay System (Promega) and GloMax system (Turner BioSystems). Relative luminescence units (RLU) were normalized to the positive control wells where cells were infected with pseudovirus in the absence of sera. The neutralization titers (ID50) were determined as the serum dilution at which infectivity was inhibited by 50%, respectively using a non-linear regression curve fit (GraphPad Prism software version 8.3). Notably, this pseudovirus neutralization assay revealed an excellent correlation with authentic virus neutralization on a panel of human convalescent sera.

Antigen Specific T Cell Assays Using Non-Human Primate Cells

To analyze the SARS-CoV-2 protein-specific T cell, 15-mer peptides (n=157 and n=158) overlapping by 11 amino acids (aa) and covering the SARS-CoV-2 Spike sequence (aa 1 to 1273) were synthesized by JPT Peptide Technologies (Berlin, Germany) and used at a final concentration of 2 μg/mL.

T-cell responses were characterized by measurement of the frequency of PBMC expressing IL-2 (PerCP5.5, MQ1-17H12, BD), IL-17a (Alexa700, N49-653, BD), IFN-γ (V450, B27, BD), TNF-α (BV605, Mab11, BioLegend), IL-13 (BV711, JES10-5 Å2, BD), CD137 (APC, 4B4, BD) and CD154 (FITC, TRAP1, BD) upon stimulation with the two peptide pools. CD3 (APC-Cy7, SP34-2, BD), CD4 (BV510, L200, BD) and CD8 (PE-Vio770, BW135/80, Miltenyi Biotec) antibodies was used as lineage markers. One million of PBMC were cultured in complete medium (RPM11640 Glutamax®+, Gibco; supplemented with 10% FBS), supplemented with co-stimulatory antibodies (FastImmune® CD28/CD49d, Becton Dickinson). The cells were stimulated with S sequence overlapping peptide pools at a final concentration of 2 μg/mL. Brefeldin A was added to each well at a final concentration of 10 μg/mL and the plate was incubated at 37° C., 5% C02 during 18 h. Next, cells were washed, stained with a viability dye (LIVE/DEAD® fixable Blue dead cell stain kit, ThermoFisher), and then fixed and permeabilized with the BD Cytofix/Cytoperm® reagent. Permeabilized cell samples were stored at −80° C. before the staining procedure. Antibody staining was performed in a single step following permeabilization. After 30 min of incubation at 4° C., in the dark, cells were washed in BD® Perm/Wash buffer then acquired on the LSRII cytometer (Beckton Dickinson). Analyses were performed with the FlowJo® v.10 software. Data are presented as the sum of each peptide pool and the non-stimulated (NS) condition was multiplied by two.

Statistical Analysis

Statistical significance between groups was performed using Graphpad Prism (v9.2.0). Differences between unmatched groups were compared using an unpaired Mann-Whitney U test (significance p<0.05), and differences between matched groups were compared using Wilcoxon signed-rank test (p<0.1). Statistical analysis of NHP gRNA and sgRNA were carried out using Mann-Whitney unpaired t-test in GraphPad Prism software (v8.3.0).

Results

S-LV Formation and Characterization

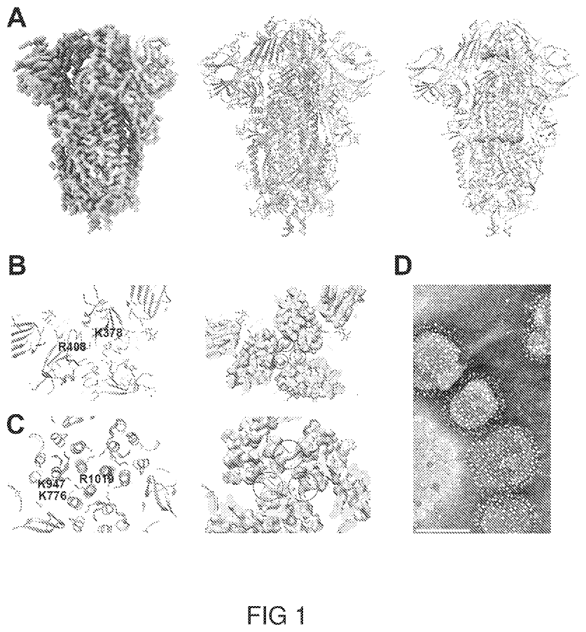

The S glycoprotein construct ‘2P’ was expressed in mammalian cells and purified by Ni²⁺-affinity and size exclusion chromatography (SEC), with yields up to 10 mg/liter using Expi293F cells. This produced native trimers as determined by negative staining electron microscopy and 2-D class averaging of the single particles. Chemical cross-linking with 4% formaldehyde (FA) produced preserved the native structure of the S trimer over longer time periods than native S trimer by increasing the thermostability to a Tm of 65° C. The cryo-electron microscopy structure of FA-cross-linked S (FA-S) at 3.4 Å resolution revealed two major sites of cross linking, as shown in (A) in FIG. 1. RBD residues R408 and K378 cross-linked neighboring RBDs producing S trimers in the closed “RBD-down” conformation as shown in (A) and (B) in FIG. 1. The second site introduced inter S2 subunit bonds by cross-linking R1019 of the central S2 helix and/or S2 K776 with S2 HR1 K947 as shown in (C) in FIG. 1. FA-S was incubated with liposomes (Phosphatidylcholine 60%, Cholesterol 36%, DGS-NTA 4%), and efficiently captured via its C-terminal His-tag. Free, unbound Fa-S was removed from the S proteoliposomes by sucrose gradient centrifugation and decoration of the liposomes with FA-S(S-LV) was confirmed by negative staining electron microscopy as shown in (D) in FIG. 1.

FA-S-LVs Immunization Induced Potent Neutralizing Antibody Responses in Cynomolgus Macaques

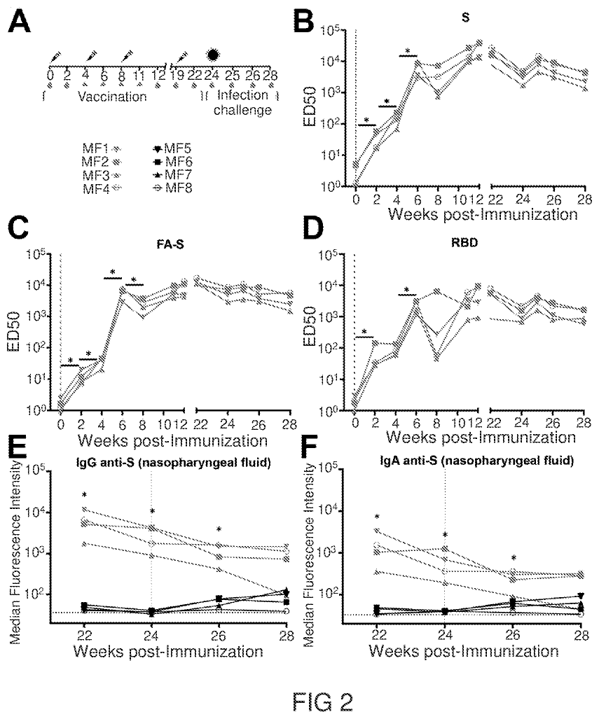

FA-S-LVs were produced for a small vaccination study of cynomolgus macaques to evaluate immunogenicity and elicitation of neutralizing antibodies. Four cynomolgus macaques were immunized with 50 μg S-LVs adjuvanted with monophospholipid A (MPLA) liposomes by the intramuscular route at weeks 0, 4, 8 and 19, as illustrated in (A) in FIG. 2. Sera of the immunized macaques were analysed for binding to native S glycoprotein (S), FA cross-linked S glycoprotein (FA-S) and RBD in two weeks intervals. The results revealed similar S-specific Ab titers for all animals. S ED50 titers increased from about 75 on week 4 to about 10000 on week 6 and to about 20000 on week 12, after the first, second and third immunization, respectively, as shown in (B) in FIG. 2. Slight reductions in titers were detected against FA-S, as shown in (C) in FIG. 2. Titers against RBD alone reached ID50s of about 100 on week 4, about 4500 on week 6 and slight increases on week 12 for some animals, as shown in (D) in FIG. 2. This shows that the first and second immunization induced significant RBD titers, while the third immunization boosted non-RBD antibodies since the week 12 S-specific titers were more than 4 times higher than the RBD-specific titers in contrast to previous time points at which this ratio was lower. A fourth immunization did not further boost antibody generation and titers at week 22 were lower or comparable to week 12 titers. These results demonstrate that FA-S-LV immunization induced primarily RBD-specific antibodies after the first and second immunization, while the third immunization increased the generation of non-RBD antibodies significantly.

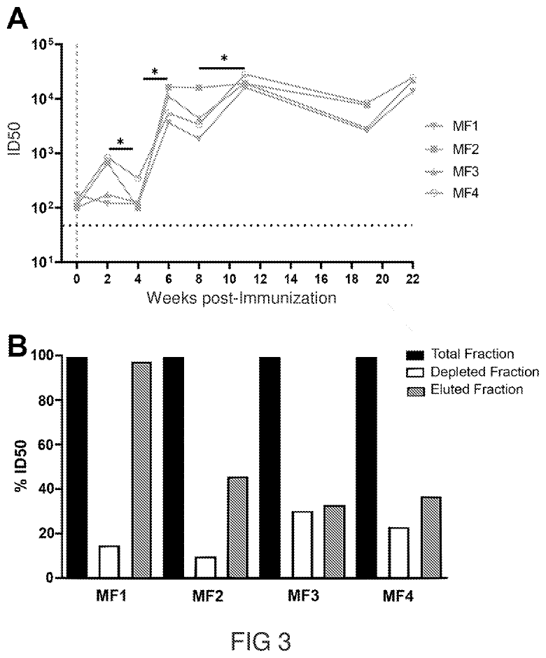

Serum neutralization titers using wild-type pseudovirus were elicited in all four animals. At week 2 after the first immunization, a ID50 titer between 100 and 1000 was observed, which dropped close to baseline at week 4, but was significantly increased at week 6, two weeks after the second immunization demonstrating ID50s between 5000 and about 20000. The ID50s then decreased at week 8 and increased to 20000 to about 40000 at week 11, three weeks after the third immunization. At week 19, neutralization potency decreased but was still high, indicating that three immunizations induced robust neutralization titers. The fourth immunization boosted neutralization titers to the same level as after the third immunization, as shown in (A) in FIG. 3.

The serum at week 11 was depleted by anti-RBD affinity chromatography, resulting in no detectable RBD antibodies by ELISA. RBD-specific Ab-depleted serum showed 10 to 30% neutralization compared to the complete serum, indicating some level of non-RBD specific neutralization. While RBD-specific Ab neutralization largely dominated in two animals, the fraction of non RBD-specific Ab neutralization activity, as shown in (B) in FIG. 3, appeared greater in the other two animals, suggesting a participation of these Abs in the high neutralization titers.

FA-S-LV Immunization Protected Cynomolgus Macaques from SARS-CoV-2 Infection

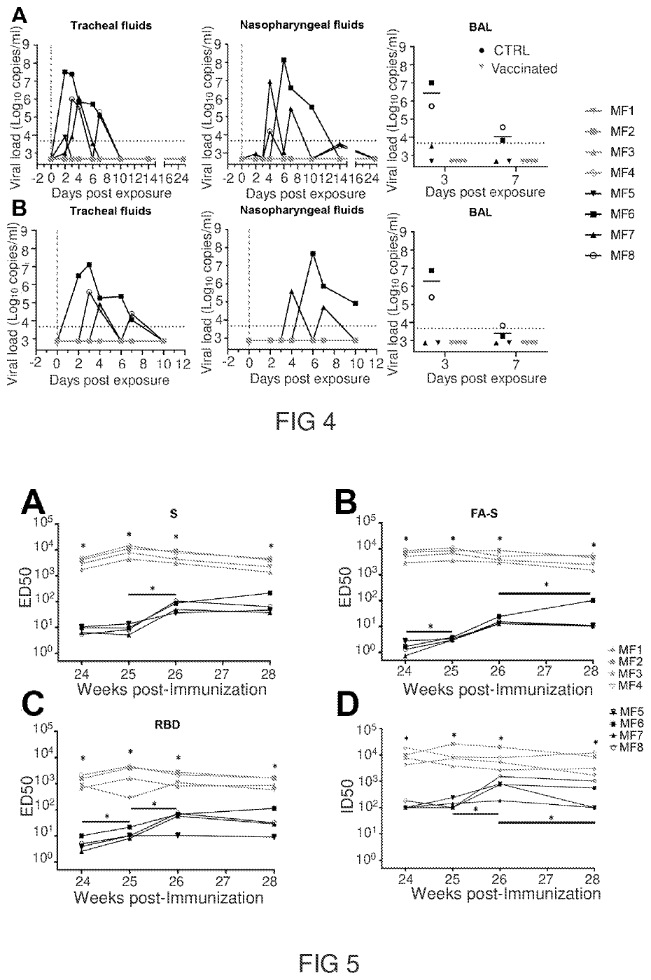

In order to determine the extent of FA-S-LV vaccination induced protection, vaccinated and non-vaccinated animals (n=4) were infected with the primary SARS-CoV-2 isolate (BetaCoV/France/IDF/0372/2020) with a total dose of 1×10⁵ plaque forming units (pfu). Infection was induced by combining intranasal (0.25 mL into each nostril) and intratracheal (4.5 mL) routes at week 24, 5 weeks after the last immunization. Viral load in the control animal group peaked in the trachea at 3 days post-exposure (dpe) with a median value of 6.0 log₁₀ copies/ml and in the nasopharynx between days 4 and 6 pe with a median copy number of 6.6 log₁₀ copies/ml, as shown in (A) in FIG. 4. Viral loads decreased subsequently and no virus was detected on day 10 dpe in the trachea, while some animals showed viral detection up to day 14 dpe in the nasopharyngeal swabs. In the bronchoalveolar lavage (BAL), three CTRL animal out of four showed detectable viral loads at day 3 pe, and two of them remained detectable at day 7 dpe with mean value of 5.4 and 3.6 log₁₀ copies/mL respectively. Rectal fluids tested positive in one animal, which also had the highest tracheal and nasopharyngeal viral loads. Viral subgenomic RNA (sgRNA), which is believed to estimate the number of infected and productively infected cells collected with the swabs or during the lavage, showed peak copy numbers between day 3/4 and 6 pe in the tracheal and nasopharyngeal fluids, respectively, as shown in (B) in FIG. 4. In the BALs, the two animals presenting high genomic viral loads also showed detectable sgRNA at days 3 and 7 pe, with medians of 5.1 and 3.1 log₁₀ copies/mL respectively.

In contrast to control animals, neither gRNA nor sgRNA was detected at any point in the vaccinated group. The mean gRNA peaks in the trachea and nasopharynx (6.0 and 6.6 log₁₀ copies/mL respectively) of the control group were higher (p=0.0286) than those of the vaccinated group. The area under the curve was also higher in the trachea of the control group (6.2 log₁₀, p=0.286). In the BAL, the difference was not statistically significant due to the low number of animals. The complete absence of viral RNA in the vaccinated group, both in the upper and lower respiratory tract, strongly suggested that sterilizing immunity was induced by vaccination. ID50 antibody titers against S, FA-S and RBD decreased slightly from the day of infection (week 24) to 4 weeks post exposure (pe), as shown in (A), (B) and (C) in FIG. 5, although a small increase in Ab titers is observed at week 1 pe (week 25). Ab titers also correlated with a slight decrease in neutralization from week 24 to 4 weeks pe, although one animal showed a small increase in neutralization on week 25, 1 week pe, as shown in (D) in FIG. 5. This demonstrated that challenge of vaccinated animals did not significantly boost their immune system. In contrast, the control group started to show clear detection of S, FA-S and RBD-specific IgG on week 2 pe (week 26), which correlated with the detection of neutralization on week 2 pe in most animals. Protection of vaccinated animals further correlated with the presence of significant S and RBD-specific IgG and IgA in nasopharyngeal fluids as shown in (E) in FIG. 2. This indicated that S-LV vaccination induced mucosal immunity that very likely contributed to the sterilizing effect of vaccination.

During the first 14 dpe, all control animals showed mild pulmonary lesions characterized by nonextended ground-glass opacities (GGOs) detected by chest computed tomography (CT). Vaccinated animals showed no significant impact of challenge on CT scores. The only animal showing a lesion score >10 was in the control group. Whereas all control animals experienced monocytoses between days 2 and 8 pe, probably corresponding to a response to infection, monocyte counts remained stable after challenge for the vaccinated monkeys, in agreement with the absence of detectable anamnestic response in the latter animals.

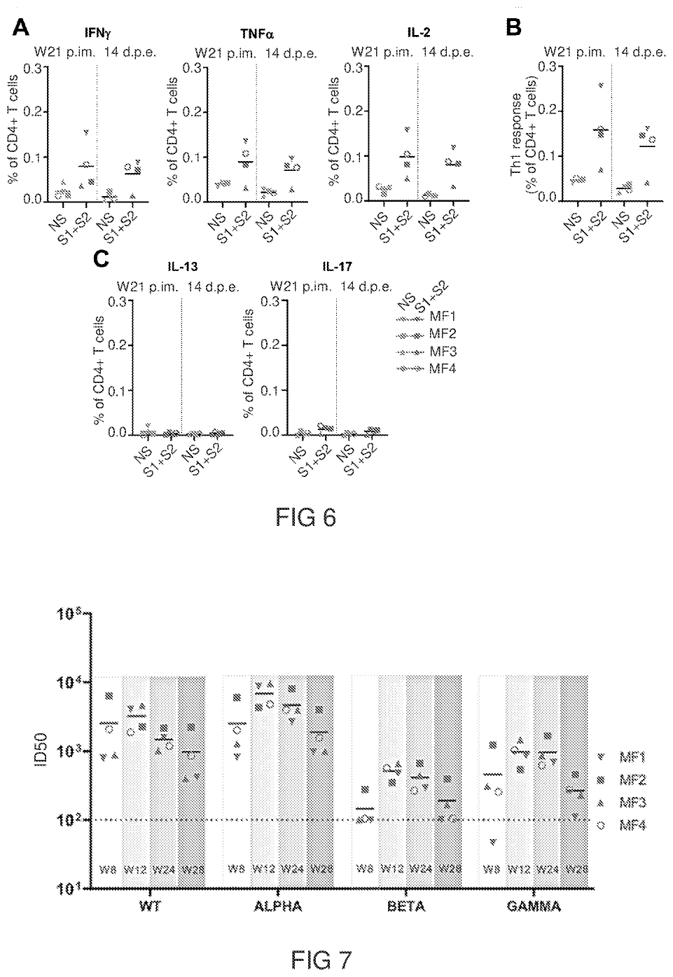

The levels of CD4 and CD8 specific T cells were measured in both groups of animals. Before exposure, Th1 type CD4+ T-cell responses were observed in all vaccinated macaques following ex vivo stimulation of PBMCs with S-peptide pools, as shown in FIG. 6. None had detectable anti-S CD8+ T cells. No significant difference was observed at day 14 pe, also in agreement with the absence of an anamnestic response in vaccinated animals. In contrast, the anti-S Th1 CD4+ response increased post exposure for most of the control animals. These results demonstrate that FA-S-LV vaccination can produce sterilizing immunity indicating that such a vaccination scheme would be efficient to interrupt the chain of SARS-CoV-2 transmission.

FA-S-LVs Vaccination Generated Robust Neutralization of SARS-CoV-2 Variants

Serum neutralization was further tested against variants B.1.1.7 (Alpha, UK), B.1.351 (Beta, SA) and P.1 (Gamma, BR). Comparing the sera of the vaccinated and the non-vaccinated group at weeks 24 and 28 showed high neutralization titers for all three variants with median ID50s ranging from 10.000 to 20.000, comparable to wild-type pseudovirus neutralization. However, since the background of pre-exposure serum neutralization of the non-vaccinated challenge group was relatively high (median ID50s ranging from 400 of 1100), the neutralization with purified IgG from serum samples of the vaccinated group from week 8 (after 2 immunizations), week 12 (3 immunizations) and weeks 24 and 28 (4 immunizations) was repeated. This showed median ID50s of about 4500 for wild type (WT) and Alpha on week 8, as shown in FIG. 7, comparable to WT serum neutralization (FIG. 3, (A)). Lower ID50s were observed against Beta and Gamma at week 8, respectively. Neutralization potency was increased after the third immunization (week 12) with median ID50s of about 5000 (WT), about 8000 (Alpha), about 800 (Beta) and 1000 (Gamma). Neutralization titers did not increase after the fourth immunization at week 24 and started to decrease at week 28, as shown in FIG. 7. These results demonstrate that three immunizations provided robust protection against the variants.

1. A recombinant SARS-CoV-2 S glycoprotein ectodomain trimer comprising three recombinant protomers each containing at least the SARS-CoV-2 S glycoprotein ectodomain, wherein:
in each protomer, the furin cleavage site, situated at positions 682 to 685 in the amino acid sequence of the native SARS-CoV-2 S glycoprotein (SEQ ID No: 1), is inactivated/disrupted;
the amino acid residue at position 408 in the amino acid sequence of the native SARS-CoV-2 S glycoprotein (SEQ ID No: 1) of one of said protomers is covalently linked to the amino acid residue at position 378 in the amino acid sequence of the native SARS-CoV-2 S glycoprotein (SEQ ID No: 1) of another one of said protomers; and
the amino acid residue at position 947 in the amino acid sequence of the native SARS-CoV-2 S glycoprotein (SEQ ID No: 1) of one of said protomers is covalently linked to the amino acid residue at position 1019 in the amino acid sequence of the native SARS-CoV-2 S glycoprotein (SEQ ID No: 1) of another one of said protomers and/or the amino acid residue at position 947 in the amino acid sequence of the native SARS-CoV-2 S glycoprotein (SEQ ID No: 1) of one of said protomers is covalently linked to the amino acid residue at position 776 in the amino acid sequence of the native SARS-CoV-2 S glycoprotein (SEQ ID No: 1) of another one of said protomers.

2. The trimer as claimed in claim 1, wherein in each protomer the amino acid residues situated at positions 682 to 685 in the amino acid sequence of the native SARS-CoV-2 S glycoprotein (SEQ ID No: 1) are substituted by an amino acid motif of sequence GSAS (SEQ ID No: 2).

3. The trimer as claimed in claim 1, wherein each protomer is linked to a C-terminal trimerization domain.

4. The trimer as claimed in claim 1, wherein in each protomer the amino acid residue at position 408 in the amino acid sequence of the native SARS-CoV-2 S glycoprotein (SEQ ID No: 1) is an arginine residue, the amino acid residue at position 378 in the amino acid sequence of the native SARS-CoV-2 S glycoprotein (SEQ ID No: 1) is a lysine residue, and said arginine residue of one of said protomers and said lysine residue of another one of said protomers are linked by a methylene bridge.

5. The trimer as claimed in claim 1, wherein in each protomer the amino acid residue at position 947 in the amino acid sequence of the native SARS-CoV-2 S glycoprotein (SEQ ID No: 1) is a lysine residue, the amino acid residue at position 1019 in the amino acid sequence of the native SARS-CoV-2 S glycoprotein (SEQ ID No: 1) is an arginine residue, and said lysine residue of one of said protomers and said arginine residue of another one of said protomers are linked by a methylene bridge.

6. The trimer as claimed in claim 1, wherein in each protomer the amino acid residue at position 947 in the amino acid sequence of the native SARS-CoV-2 S glycoprotein (SEQ ID No: 1) is a lysine residue, the amino acid residue at position 776 in the amino acid sequence of the native SARS-CoV-2 S glycoprotein (SEQ ID No: 1) is a lysine residue and said lysine residues are linked by a methylene bridge.

7. The trimer as claimed in claim 1, wherein each protomer comprises at least two proline substitutions at positions 986 and 987 of the amino acid sequence of the native SARS-CoV-2 S glycoprotein (SEQ ID No: 1).

8. The trimer as claimed in claim 1, wherein each protomer is linked to at least one tag at its C-terminal end.

9. The trimer as claimed in claim 1, wherein each protomer comprises the 1208 first amino acid residues of the SARS-CoV-2 S glycoprotein or a protein having at least 90% amino acid sequence identity therewith.

10. The trimer as claimed in claim 1, which is a homomeric trimer.

11. A method of producing the trimer as claimed in claim 1, comprising:
expressing nucleic acid molecule(s) encoding said recombinant protomer(s) in a host cell to produce said trimer,
purifying said trimer, and
treating said trimer with formaldehyde.

12. A proteoliposome comprising a lipid vesicle a surface of which is coated by the trimer as claimed in claim 1.

13. The proteoliposome as claimed in claim 12, wherein said lipid vesicle comprises 60% by weight of L-α-phosphatidylcholine, 36% by weight of cholesterol and 4% by weight of a polyhistidine-tag conjugating lipid.

14. A method of preparing the proteoliposome as claimed in claim 12, comprising incubating said trimer with said lipid vesicle.

15. A vaccine comprising proteoliposomes as claimed in claim 12.

16. The vaccine as claimed in claim 15, in a unit dose comprising 50 to 100 μg of said proteoliposomes.

17. A method of treating or preventing a SARS-CoV-2 infection in a subject, comprising administering to the subject a therapeutically effective amount of the vaccine as claimed in claim 15.

18. The method as claimed in claim 17, comprising administering a therapeutically effective amount of the vaccine at least twice to the subject.

19. The method as claimed in claim 17, comprising administering a therapeutically effective amount of the vaccine at least three times to the subject.

20. The method as claimed in claim 17, wherein the vaccine is administrated to the subject intramuscularly or intranasally.
